(19) United States
(12) Patent Application Publication
(10) Pub. No.: US 2017/0065520 A1
(43) Pub. Date: Mar. 9, 2017

(54) STABLE LIPOSOMAL FORMULATIONS OF RAPAMYCIN AND RAPAMYCIN DERIVATIVES FOR TREATING CANCER

(71) Applicant: Manli International Ltd, Singapore (SG)

(72) Inventors: Ting-Bin Yu, Irvine, CA (US); Zhiwei Shi, Irvine, CA (US); Shih-Horng Su, Irvine, CA (US)

(21) Appl. No.: 14/849,100

(22) Filed: Sep. 9, 2015

(51) Int. Cl. A61K 9/127; A61K 31/439

(52) U.S. Cl. CPC A61K 9/1271; A61K 31/439; A61K 9/1278

(57) ABSTRACT

A stable liposomal formulation for treating cancer. The formulation includes liposomes containing one or more of POPC, DMPC, and DOPC and one or more of the anti-cancer drugs sirolimus, umirolimus, and everolimus encapsulated in the liposomes. Also provided are efficient remote film loading methods for loading the liposomes with the anti-cancer drugs and methods for treating cancer with the liposomal formulations.

### BACKGROUND OF THE INVENTION

Field

The application relates to the field of anti-cancer drugs, in particular, methods for loading hydrophobic anti-cancer drugs into liposomes and for treatment of cancers with the liposomes.

Background

Rapamycin, also known as sirolimus, is a macrolide antibiotic initially developed for use as an immune suppressor for transplant patients. Subsequently, it was used as a drug coating for coronary artery stents, where it functions to reduce restenosis following angioplasty by inhibiting smooth muscle cell proliferation.

Sirolimus and derivatives of this drug have also been found to be effective for treating certain cancers. For example, sirolimus has anti-tumor activity. See U.S. Pat. No. 4,885,171. Everolimus, the 40-O-(2-hydroxyethyl) derivative of sirolimus, has been approved for treating advanced kidney cancer, advanced hormone receptor-positive/HER2-negative breast cancer, and pancreatic neuroendocrine tumors.

It has been demonstrated that umirolimus i.e., 40-alkoxyalkyl-rapamycin, and umirolimus-loaded polymer micelles can both inhibit cancer cell growth in vitro and the micelles are effective for slowing the growth of experimental tumors in vivo. See US Patent Application Publication 2014/0154305. The polymer micelle encapsulation of umirolimus significantly improves the solubility and stability of this drug and results in its sustained delivery.

As an alternative to polymer micelles, liposomes have been employed to improve drug delivery of sirolimus and its derivatives. For example, sirolimus, everolimus, and tacrolimus have been encapsulated in liposomes using two passive loading methods, namely, thin-film hydration and ethanol injection. However, due to low solubility and hydrophobicity of these drugs, the amount of drug encapsulated and drug encapsulation efficiency is particularly low, i.e., <0.5 mg/mL and <90%, respectively. Drug leakage from the liposomes also occurs with passive loading techniques.

A remote film loading technique that requires steps of drug dissolution and solvent removal has been used to entrap sirolimus into pre-formed liposomes. Although the method results in high drug encapsulation efficiency, there are potential risks to drug stability during the loading procedure.

Thus, the need exists for developing new liposomal loading methods for hydrophobic drugs to improve drug content, drug to lipid ratio, and drug encapsulation efficiency. Such methods should form drug-loaded liposomes having a higher therapeutic index as compared to existing liposomes, particularly for umirolimus and other rapamycin-derivatives.

### SUMMARY OF THE INVENTION

To satisfy this need, a stable liposomal formulation for treating cancer is provided. The stable formulation includes a liposome that contains at least one lipid bilayer formed of one or more phosphatidylcholine selected from palmitoyloleoylphosphatidylcholine (POPC), dimyristoylphosphatidylcholine (DMPC), and dioleoylphosphatidylcholine (DOPC). The liposome has a diameter of 50 nm to 2 μm and is free of cholesterol. Encapsulated in the liposome is an anti-cancer drug selected from sirolimus, umirolimus, and everolimus.

Also provided is a method for loading a hydrophobic drug into liposomes. The method includes the steps of (i) obtaining cholesterol-free liposomes having at least one lipid bilayer, (ii) adding the cholesterol-free liposomes to an aqueous solution to form a suspension such that there is substantially no transmembrane potential across the lipid bilayer, (iii) adding a hydrophobic drug in the absence of a solubility enhancer to the suspension to form a mixture, and (iv) stirring the mixture for 4 to 48 hours at room temperature. The method results in loading of at least 80% of the hydrophobic drug into the liposomes. In an embodiment, the method consists of the steps set forth in this paragraph.

Additionally, a method for preparing a hydrophobic drug encapsulated in a cholesterol-free liposome is disclosed. The method is carried out by (i) suspending one or more of POPC, DMPC, and DOPC in an aqueous buffer to form a lipid suspension, (ii) stirring the lipid suspension for at least 30 minutes at room temperature to form multilamellar vesicles (MLVs), (iii) extruding the MLVs to form large unilamellar vesicles (LUVs) having a diameter of 50 nm to 2 μm, (iv) adding the LUVs to an aqueous solution to form a suspension such that there is substantially no transmembrane potential across the LUVs, (v) adding a hydrophobic drug to the suspension in the absence of a solubility enhancer to form a mixture, (vi) stirring the mixture for 4 to 48 hours at room temperature to form a drug-loaded liposome suspension, and (vii) filtering the drug-loaded liposome suspension to remove unencapsulated hydrophobic drug. The method allows for encapsulation of at least 80% of the added hydrophobic drug. In another embodiment, the method consists of the steps set forth in this paragraph.

A method for treating cancer is also disclosed. The method requires the steps of administering to a subject in need thereof an effective amount of the stable liposomal formulations described above. The effective amount is sufficient to inhibit growth of cancer cells in the subject.

The details of one or more embodiments of the invention are set forth in the description, in the drawings, and in the examples below. Other features, objects, and advantages of the invention will be apparent from the detailed description, the drawings, and also from the claims. All publications and patent documents cited herein are incorporated by reference in their entirety.

### BRIEF DESCRIPTION OF THE DRAWINGS

The invention description below refers to the accompanying drawings, of which:

FIG. 1 is a plot of the in vitro drug release profile of umirolimus (URL) and liposomal umirolimus (LipoURL);

FIG. 2 is a plot of the in vitro drug release profile of everolimus (ERL) and liposomal everolimus (LipoERL); and

FIG. 3 is a plot of the in vitro drug release profile of sirolimus (SRL), and liposomal sirolimus (LipoSRL).

### DETAILED DESCRIPTION

As mentioned above, a stable liposomal formulation for treating cancer is disclosed. The liposomes in the formulation contain at least one lipid bilayer formed of a phosphatidylcholine selected from POPC, DMPC, and DOPC, or mixtures of these three phosphatidylcholines, and are free of cholesterol. In a particular aspect, the liposomes contain one lipid bilayer. In another aspect, the liposomes contain only POPC, DMPC, or DOPC. In a preferred embodiment, the liposomes contain only POPC.

Alternatively, the lipid bilayer of the liposomes includes the phosphatidylcholine together with a phospholipid conjugated to a polyethylene glycol (PEG) moiety. The PEG-conjugated phospholipid can be 1,2-distearoyl-sn-glycero-3-phosphoethanolamine-N-[amino(PEG)](DSPE-PEG); 1,2-dioleoyl-sn-glycero-3-phosphoethanolamine-N-[methoxy(PEG)](DOPE-PEG); 1,2-dipalmitoyl-sn-glycero-3-phosphoethanolamine-N-[methoxy(PEG)](DPPE-PEG); 1,2-dimyristoyl-sn-glycero-3-phosphoethanolamine-N-[methoxy(PEG)](DMPE-PEG); or mixtures thereof. In a particular embodiment, the PEG-conjugated phospholipid is DSPE-PEG.

When the liposome includes both the phosphatidylcholine and the PEG-conjugated phospholipid, the weight ratio between them can be 5:1 to 100:1, e.g., 5:1, 7.5:1, 10:1, 15:1, 20:1, 30:1, 40:1, 50:1, 60:1, 70:1, 80:1, 90:1, and 100:1. A preferred ratio is 10:1.

The molecular weight of the PEG moiety that is conjugated to the phospholipid can be 150 to 3000 g/mol, e.g., 150, 200, 250, 300, 350, 500, 750, 1000, 1250 1500, 1750, 2000, 2250, 2500, 2750, 3000 g/mol. In a preferred embodiment, the molecular weight of the PEG moiety is 2000 g/mol.

The liposomes can have a diameter of 50 nm to 2 μm (e.g., 50 nm, 100 nm, 150 nm, 200 nm, 250 nm, 500 nm, 1 μm, 1.5 μm, and 2 μm). In an embodiment, the liposomes have a diameter of 50 nm to 500 nm. In a preferred embodiment, the diameter is 100 nm.

The liposomes in the stable liposomal formulation encapsulate a hydrophobic drug for treating cancer. The hydrophobic drug can be an anti-proliferative drug, e.g., sirolimus, umirolimus, or everolimus. In a particular formulation, the hydrophobic drug is umirolimus.

The weight ratio between the hydrophobic drug and the phosphatidylcholine component of the liposomes can be 1:5 to 1:100 (e.g., 1:5, 1:10, 1:15, 1:20, 1:25, 1:30, 1:35, 1:40, 1:45, 1:50, 1:75, and 1:100). In an embodiment, the drug to phosphatidylcholine weight ratio is 1:10. In another particular embodiment, the weight ratio is 1:20.

The concentration of the hydrophobic drug in the stable liposomal formulation can be 0.01 mg/mL to 10 mg/mL. For example, the drug concentration in the formulation can be 0.01 mg/mL, 0.05 mg/mL, 0.5 mg/mL, 1.0 mg/mL, 2.5 mg/mL, 5.0 mg/mL, and 10 mg/mL. In a particular embodiment, the drug concentration is 1 mg/mL.

Additionally, the stable liposomal formulation can have a pH of 6.0 to 8.0. In a preferred embodiment, the pH is 7.4.

In a particular aspect, the stable liposomal formulation for treating cancer includes liposomes formed only of POPC and DSPE-PEG 2000, the liposomes having umirolimus encapsulated therein. The liposomes are cholesterol-free, have a diameter of about 100 nm, and the weight ratio between the umirolimus and the POPC is 1:20. This specific formulation contains 1 mg/mL of umirolimus and has a pH of 7.4.

The stable liposomal formulation also improves the stability of the hydrophobic drug in solution. For example, the hydrophobic drug in the formulation can be stable for 7-14 days when stored at 5° C. as compared to the drug in an aqueous suspension. In this context, stability is defined as a loss of no more than 5% of the starting amount of drug in the formulation.

Advantageously, the hydrophobic drug can be released from the liposomes in the formulation over an extended period of time after administration. That is to say, the formulation is a sustained release formulation. For example, the hydrophobic drug can be released from the liposomes after administration of the formulation continuously over a period of up to 3 months, e.g., 7, 14, 21 days and 1, 2, and 3 months.

Also mentioned above, a method for loading a hydrophobic drug into liposomes is provided. The method is an improved remote film loading technique in which the liposomes are obtained first and the hydrophobic drug is then loaded into the liposomes.

The liposomes for use in the loading method are cholesterol-free, have at least one lipid bilayer that contains one or more of POPC, DMPC, and DOPC, and have a diameter of 50 nm to 2 μm. In an embodiment, the liposomes contain only POPC.

Alternatively, the cholesterol-free liposomes have at least one lipid bilayer that contains one or more of POPC, DMPC, and DOPC and also contains a PEG-conjugated phospholipid selected from DSPE-PEG, DOPE-PEG, DPPE-PEG, and DMPE-PEG.

The molecular weight of the PEG moiety that is conjugated to the phospholipid can be 150 to 3000 g/mol, e.g., 150, 200, 250, 300, 350, 500, 750, 1000, 1250 1500, 1750, 2000, 2250, 2500, 2750, 3000 g/mol. In a preferred embodiment, the molecular weight of the PEG moiety is 2000 g/mol.

In a particular aspect, the liposomes contain only POPC. In another embodiment, the liposomes contain only POPC and DSPE-PEG2000.

The liposomes can be obtained by forming MLVs that contain one or more of POPC, DMPC, DOPC, and, optionally, DSPE-PEG2000, and extruding the MLVs to obtain cholesterol-free liposomes having a diameter of 50 nm to 2 μm. More specifically, one or more of POPC, DMPC, DOPC, and, optionally, DSPE-PEG2000 are suspended in an aqueous buffer to form a lipid suspension, the suspension is stirred for at least 30 minutes at room temperature to form MLVs.

The MLVs are converted into large unilamellar vesicles (LUVs) by an extrusion process. For example, the MLVs can be extruded through a 3-layered polycarbonate filter from 3 to 20 times. In a preferred extrusion process, the MLVs are extruded 10 times.

The polycarbonate filter can have a pore size ranging from 50nm to 200 nm. In a particular embodiment, the pore size is 100 nm. Again, the resulting LUVs, i.e., liposomes, can have a diameter of 50 nm to 2 μm.

The cholesterol-free liposomes described above are then added to an aqueous solution to form a suspension. Importantly, the aqueous solution employed should be the same solution or similar to that used for producing the cholesterol-free liposomes such that there is substantially no transmembrane potential across the lipid bilayer of the liposomes. For example, the ionic strength, pH, and osmolarity should be closely matched such that there is no ionic gradient, pH gradient, or osmotic gradient across the liposomal membrane. This can be ensured, e.g., by using PBS to form the cholesterol-free liposomes and also diluting them in PBS to form the suspension.

In this context, the phrase “substantially no transmembrane potential” means a level of transmembrane potential below which a drug would not be actively loaded into the liposome. For example, see Akbarzadeh et al., Nanoscale Research Letters 2013, 8:102.

A hydrophobic drug is added to the suspension of liposomes in the aqueous solution to form a mixture. The hydrophobic drug can be sirolimus, umirolimus, or everolimus. In a particular method, the drug is umirolimus The loading method at this stage does not require the use of a solubility enhancer, e.g. a cyclodextrin, to solubilize the hydrophobic drug in the aqueous solution. Indeed, this is not necessary or desirable.

Not to be bound by theory, it is believed that the hydrophobic drug, as it is added to the liposomes suspended in the aqueous solution, interact strongly with lipid tails of the liposomes due to the hydrophobic nature of the drug, leading to its encapsulation inside the lipid bilayer of the liposome.

The mixture of liposomes and hydrophobic drug is stirred for 4 to 48 hours at room temperature, resulting in at least 80% (e.g., 80%, 85%, 90%, 95%, and 100%) of the added hydrophobic drug loaded into the cholesterol-free liposomes.

Also within the scope of the application is a method for treating cancer using the stable liposomal formulations described above. The method requires administering to a cancer patient an effective amount of the stable liposomal formulation that contains an anti-cancer drug selected from sirolimus, umirolimus, or everolimus. The effective amount inhibits growth of cancer cells in the patient.

A skilled person in the art can readily determine the effective amount of the stable liposomal formulation that should be administered to the cancer patient. For example, response to drug dose over time can be followed by measuring tumor size by MRI or CT scan.

The stable liposomal formulations can be administered to a cancer patient via any conventional method, including, but not limited to, intraperitoneal injection, intravenous injection, direct injection into a tumor, injection into the arterial circulation upstream of a tumor, and nasal inhalation. The stable liposomal formulations described above can also be formed into a pill or a capsule for oral administration.

The types of cancer that can be treated by administering the above-described stable liposomal formulations include but are not limited to acute lymphocytic leukemia, acute myeloid leukemia, adrenal cancer, adult soft tissue sarcoma, anal cancer, aplastic anemia, basal and squamous cell skin cancer, bile duct cancer, bladder cancer, bone cancer, brain/CNS tumors, breast cancer, breast cancer in man, cancer in children, cancer of unknown primary, Castleman's disease, cervical cancer, chronic lymphocytic leukemia, chronic myeloid leukemia, chronic myelomonocytic leukemia, colorectal cancer, endometrial cancer, esophagus cancer, Ewing Family of tumors, eye cancer, gallbladder cancer, gastric cancer, gastrointestinal carcinoid, gastrointestinal stromal tumor, gestational trophoblastic disease, Hodgkin's disease, Kaposi's sarcoma, kidney cancer, laryngeal and hypopharyngeal cancer, leukemia in children, liver cancer, lung cancer-non small cell, lung cancer-small cell, Lung carcinoid tumor, malignant mesothelioma, melanoma skin cancer, multiple myeloma, myelodysplastic syndrome, nasal cavity and paranasal sinus cancer, nasopharyngeal cancer, neuroblastoma, non-Hodgkin's lymphoma, oral cavity and oropharangeal cancer, osteosarcoma, ovarian cancer, pancreatic cancer, penile cancer, pituitary tumors, prostate cancer, renal cell carcinoma, retinoblastoma, rhabdomyosarcoma, salivary gland cancer, skin lymphoma, small intestine cancer, stomach cancer, testicular cancer, thymus cancer, thyroid cancer, uterine sarcoma, vaginal cancer, vulvar cancer, Waldenstrom's macroglobulinemia, and Wilms' tumor.

Without further elaboration, it is believed that one skilled in the art can, based on the disclosure herein, utilize the present invention to its fullest extent. The following specific examples are, therefore, to be construed as merely descriptive, and not limitative of the remainder of the disclosure in any way whatsoever.

### EXAMPLES

### Example 1

### Preparation of Umirolimus-Loaded Liposomes

Four 200 mL batches of liposomes were prepared, each containing a different phosphatidylcholine, namely, EggPC, POPC, DMPC, or DOPC. Briefly, 6000 mg of each phosphatidylcholine was added to 200 mL of phosphate buffered saline (PBS) in separate 500 mL depyrogenated glass bottles. The mixtures were stirred at room temperature for at least 30 min. to form multilamellar vesicles (MLVs). The sizes of the MLVs were reduced by extrusion through a 3-stack of polycarbonate filter membranes (pore size 100 nm) using a bench top extruder (Northern Lipids Inc., Canada). After 10 extrusion passes, large unilamellar vesicles (LUVs) with an average size of ˜100 nm were obtained.

To load umirolimus into the LUVs, 50 mg of the drug was added to each of four 50 mL depyrogenated glass bottle together with 10 mL of one of the four LUV preparations per bottle. The glass bottle was capped and placed in a water bath at a temperature of 25° C. The mixture was stirred for 24 hours. The resulting solution of umirolimus-loaded liposomes was filtered through a polyvinylidene difluoride (PVDF) syringe filter having a 0.2 μm pore size to remove un-encapsulated umirolimus.

The umirolimus content of the liposomal solutions after loading was determined by reverse-phase HPLC. The liposomes were broken by mixing 50 pi samples of each liposomal solution with 1.0 ml of acetonitrile. A standard solution of umirolimus was prepared at 0.05 mg/mL in methanol. Samples were analyzed by HPLC and compared to the standard solution to quantify the amount of umirolimus in the liposome.

The intensity mean diameter of the liposomes and the polydispersity index (PDI) of the distribution were determined by dynamic light scattering (DLS) using a Zetasizer Nano instrument (Malvern Instruments, Great Britain). Each sample tested was diluted 1:25 in a 0.9% sodium chloride solution. Particle size measurements at a scattering angle of 172° were carried out using a scan of at least 5 min. with the following parameters: viscosity=1.0183 cp, refractive index=1.332, temperature=23° C. The size of the liposomes were measured and recorded prior to and after umirolimus loading. The drug loading results are summarized in the Table 1 below.

TABLE 1
Umirolimus Loading into Liposomes

| | Lipid type | | | |
|---|---|---|---|---|
| | EggPC | POPC | DOPC | DMPC |
| Vesicle size prior to drug | 98.0 | 96.6 | 97.6 | 88.6 |
| loading (nm) | | | | |
| PDI prior to drug loading | 0.070 | 0.039 | 0.059 | 0.072 |
| Umirolimus content (mg/mL) | 1.71 | 2.76 | 2.72 | 2.98 |
| Vesicle size at end of drug | 102.0 | 99.0 | 99.0 | 91.8 |
| loading (nm) | | | | |
| PDI at end of drug loading | 0.031 | 0.049 | 0.038 | 0.048 |
| Drug:Lipid ratio (w/w) | 1:17.5 | 1:10.9 | 1:11.0 | 1:10.1 |

The amount of umirolimus loaded into liposomes formed of DMPC, POPC, and DOPC was greater than 2.7 mg/mL. Unexpectedly, the amount of umirolimus loaded into these three liposomes was higher than that loaded into liposomes formed of EggPC.

### Example 2

### Preparation of Umirolimus-Loaded Liposomes Containing a Polyethylene Glycol-Conjugated Phospholipid

Liposomes containing a polyethylene glycol-conjugated (PEGylated) phospholipid were prepared by combining 1800 mg of POPC and 200 mg of DSPE PEG-2000 in 200 mL of PBS in a 500 mL depyrogenated glass bottle. The mixture was stirred at room temperature for at least 30 min. to form MLVs. The sizes of the MLVs were reduced by extrusion through a 3-stack of polycarbonate filter membranes (pore size 100 nm) using a bench top extruder (Northern Lipids Inc., Canada). After 10 extrusion passes, PEGylated LUVs with an average size of ˜100 nm were obtained.

To load umirolimus into the PEGylated LUVs, 50 mg of the drug was added to a 500 mL depyrogenated glass bottle together with 50 mL of the LUVs. The glass bottle was capped and placed in a water bath at a temperature of 25° C. The mixture was stirred for up to 24 hours. 1 mL samples were collected at 1, 2, 3, 4, 5, 6, and 24 h after initiation of stirring to evaluate umirolimus loading into the PEGylated liposomes. Each sample was filtered through a polyvinylidene difluoride (PVDF) syringe filter having a 0.2 μm pore size to remove un-encapsulated umirolimus. The umirolimus content of each sample was evaluated by HPLC as described above in EXAMPLE 1.

The results indicated that the percentage of umirolimus incorporated into the PEGylated liposomes after stirring for 1, 2, 3, 4, 5, 6, and 24 h was 59%, 67%, 77%, 82%, 87%, 91%, and 102%, respectively, based on the initial amount of umirolimus used for loading. Performing the above-described loading method unexpectedly resulted in loading of essentially all of the umirolimus into the PEGylated liposomes within 24 h.

The stability of the umirolimus-loaded PEGylated liposome formulation was evaluated during storage in clear glass vials at 5° C.±3° C. The sample temperature was monitored and recorded continuously to ensure constant temperature conditions. The solution was analyzed for umirolimus content, vesicle size, and PDI as described above in EXAMPLE 1 after storage for 2, 3, 4, 6, and 8 weeks. The stability results are shown in TABLE 2 below.

TABLE 2
Stability of formulations of umirolimus-
loaded PEGylated liposomes

| | Time Point | | | | | |
|---|---|---|---|---|---|---|
| | 0 weeks | | | | | |
| | (end of | | | | | |
| | drug | | | | | |
| | loading) | 2 week | 3 week | 4 week | 6 week | 8 week |
| umirolimus | 0.99 | 1.00 | 1.00 | 0.96 | 1.0 | 1.0 |
| content | | | | | | |
| (mg/mL) | | | | | | |
| vesicle size | 99.9 | 97.1 | 97.5 | 97.5 | 97.7 | 98.3 |
| (nm) | | | | | | |
| PDI | 0.062 | 0.048 | 0.058 | 0.053 | 0.050 | 0.048 |

The results indicate that the solution of umirolimus-loaded PEGylated liposomes is stable for at least 8 weeks when stored at 5° C.

### Example 3

### Measurement of Drug Encapsulation Efficiency of Umirolimus-Loaded Liposome Formulations

An assay was developed to characterize the efficiency of umirolimus encapsulation by the liposomes using a gel-filtration technique to remove free drug from the liposome solution. The drug to lipid ratio of liposomal formulations was determined before and after running them on a PD-10 cross-linked dextran gel (SEPHADEX® G-25) desalting column using the following equation:

$$Drug Encapsulation \% = \frac{\begin{matrix}Final Drug \text{:} Lipid Ratio \\ ( samples passed through PD \text{-} 10 column )\end{matrix}}{\begin{matrix}Initial Drug \text{:} Lipid Ratio \\ ( samples prior to PD \text{-} 10 seperation )\end{matrix}} \times 100 \%$$

Umirolimus-loaded POPC liposomes and umirolimus-loaded POPC PEGylated liposomes were prepared as described above in EXAMPLE 1 and EXAMPLE 2, respectively. The drug encapsulation efficiencies are shown in TABLE 3 below.

TABLE 3
Umirolimus drug encapsulation efficiency

| | Drug:Lipid | Drug:Lipid | |
|---|---|---|---|
| | ratio | ratio | Drug |
| | prior to column | after column | encapsulation |
| Formulation | separation | separation | efficiency (%) |
| Drug-loaded POPC | 0.130 | 0.125 | 95.9 |
| liposomes from | | | |
| EXAMPLE 1 | | | |
| (drug:lipid = 1:10) | | | |
| Drug-loaded PEGylated | 0.052 | 0.050 | 97.5 |
| DSPE/POPC liposomes | | | |
| from EXAMPLE 2 | | | |
| (drug:lipid = 1:20) | | | |

Surprisingly, the encapsulation efficiency of both the umirolimus-loaded POPC liposomes and the umirolimus-loaded POPC PEGylated liposomes was greater than 95%.

### Example 4

### Preparation of Sirolimus- and Everolimus-Loaded Liposomes

POPC LUVs were prepared as described above in EXAMPLE 1. 50 mg of sirolimus or everolimus were added to a 50 mL depyrogenated glass bottle together with 10 mL of the POPC LUVs. The glass bottle was capped and placed in a water bath at a temperature of 25° C. The mixture was stirred at room temperature for 24 h. The drug-loaded liposome solution was filtered through a 0.2μm PVDF syringe filter to removed un-encapsulated drug.

For both liposomal formulations, the drug content after encapsulation, the intensity mean diameter of the liposomes, and the PDI were determined as described above in EXAMPLE 1. The results are shown in Table 4 below.

TABLE 4
Drug-Loading of sirolimus and everolimus into POPC liposomes

Drug

| | Sirolimus | Everolimus |
|---|---|---|
| Vesicle Size prior to | 96.6 | 96.6 |
| Drug Loading (nm) | | |
| PDI prior to Drug | 0.039 | 0.039 |
| Loading | | |
| Drug content (mg/mL) | 1.85 | 4.65 |
| Drug:Lipid Ratio | 1:16 | 1:6.5 |
| (w/w) | | |
| Vesicle Size at end of | 96.1 | 99.5 |
| Drug Loading (nm) | | |
| PDI at end of Drug | 0.048 | 0.087 |
| Loading | | |
| Drug Encapsulation | 95.7 | 98.7 |
| Efficiency (%) | | |

The results indicated that sirolimus and everolimus were successfully encapsulated into POPC liposomes at high efficiencies.

### Example 5

### In Vitro Drug Release Study

An in vitro drug release assay was used to determine release profiles of umirolimus, sirolimus, and everolimus. Umirolimus-loaded liposomes were prepared as described above in EXAMPLE 1, and sirolimus-loaded and everolimus-loaded liposomes were prepared as described above in EXAMPLE 4.

For each drug, a control formulation was prepared containing 1.0 mg of drug in 5 mL of a solution containing 15% acetonitrile and 85% water. 5 mL of the control formulations and the liposomal formulations for each drug were loaded into separate dialysis tubes having a molecular weight cut-off of 50 kDal.

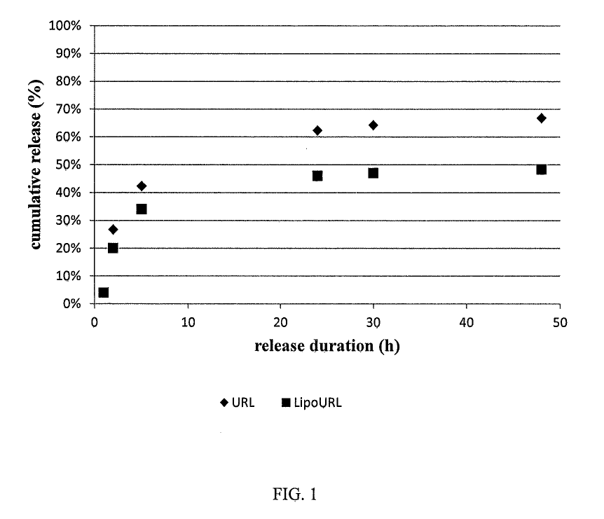

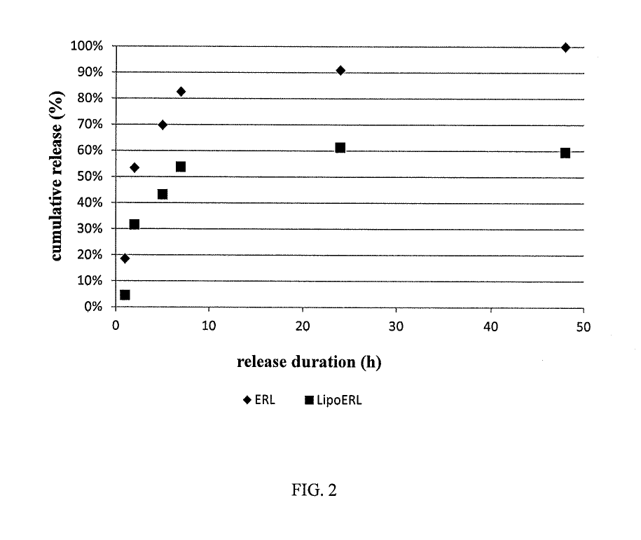

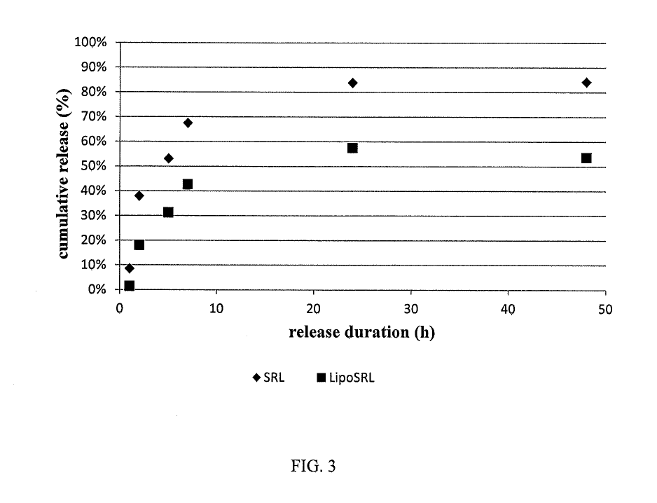

Each loaded dialysis tube was placed in an individual 50 mL tube containing 40 mL of release media (15% acetonitrile and 0.5% SDS). The release media in each tube was sampled after 1, 2, 5, 7, 24, 30, and 48 h and the drug concentration in the release media was determined by HPLC as described above in EXAMPLE 1. The cumulative drug release percentage versus release time was plotted for all samples. The results are shown in FIGS. 1, 2, and 3 for umirolimus, everolimus, and sirolimus, respectively.

The results indicated that, respectively, 67%, 100%, and 84% of the starting amounts of umirolimus, everolimus, and sirolimus diffused out of the dialysis tubing within 48 h.

By contrast, the liposome-encapsulated drugs diffused out of the dialysis tubing at a slower rate. The cumulative release of encapsulated umirolimus, everolimus, and sirolimus after 48 h was 67%, 59%, and 54%, respectively, of the starting amount of drug.

The results indicated that liposomal formulations of umirolimus, everolimus, and sirolimus are effective for sustained drug delivery.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, a person skilled in the art can easily ascertain the essential characteristics of the present invention, and without departing from the spirit and scope thereof, can make various changes and modifications of the present invention to adapt it to various usages and conditions. Thus, other embodiments are also within the claims.

1. A stable liposomal formulation for treating cancer, the stable formulation comprising a liposome that contains at least one lipid bilayer formed of a phosphatidylcholine selected from the group consisting of palmitoyloleoylphosphatidylcholine (POPC), dimyristoylphosphatidylcholine (DMPC), and dioleoylphosphatidylcholine (DOPC) or mixtures thereof; and a drug encapsulated in the liposome, wherein the drug is sirolimus, umirolimus, or everolimus; the phosphatidylcholine is free of any polyethylene glycol moieties; and the liposome has a diameter of 50 nm to 2 μm and is free of cholesterol.

2. The stable liposomal formulation of claim 1, wherein a weight ratio between the drug and the phosphatidylcholine is 1:5 to 1:100.

3. The stable liposomal formulation of claim 2, wherein the formulation contains 0.01 mg/mL to 10 mg/mL of the drug.

4. The stable liposomal formulation of claim 3, wherein the formulation has a pH of 6 to 8.

5. The stable liposomal formulation of claim 1, wherein the at least one lipid bilayer further includes a phospholipid conjugated to a polyethylene glycol (PEG) moiety.

6. The stable liposomal formulation of claim 5, wherein the phospholipid is distearoylphosphatidylethanolamine (DSPE) and the PEG moiety has a molecular weight of 150 to 3000 g/mol.

7. The stable liposomal formulation of claim 6, wherein a weight ratio between the drug and the phosphatidylcholine is 1:5 to 1:100.

8. The stable liposomal formulation of claim 7, wherein the formulation contains 0.01 mg/mL to 10 mg/mL of the drug.

9. The stable liposomal formulation of claim 8, wherein the formulation has a pH of 6 to 8.

10. The stable liposomal formulation of claim 9, wherein the phosphatidylcholine is POPC, the drug is umirolimus, the weight ratio between the umirolimus and the phosphatidylcholine is 1:20, and the formulation contains 1 mg/mL of the umirolimus.

11. A method for loading a hydrophobic drug into liposomes, the method comprising:
obtaining cholesterol-free liposomes having at least one lipid bilayer,
adding the cholesterol-free liposomes to an aqueous solution to form a suspension such that there is substantially no transmembrane potential across the at least one lipid bilayer,
adding a hydrophobic drug in the absence of a solubility enhancer to the suspension to form a mixture, and
stirring the mixture for 4 to 48 hours at room temperature, whereby at least 80% of the added hydrophobic drug is loaded into the cholesterol-free liposome.

12. The method of claim 11, wherein the hydrophobic drug is sirolimus, umirolimus, or everolimus.

13. The method of claim 12, wherein the cholesterol-free liposomes have a diameter of 80 nm to 2 μm and are obtained by forming multilamellar vesicles (MLVs) that contain one or more of palmitoyloleoylphosphatidylcholine (POPC), dimyristoylphosphatidylcholine (DMPC), dioleoylphosphatidylcholine (DOPC); and extruding the MLVs, thereby obtaining the cholesterol-free liposomes having a diameter of 50 nm to 2 μm.

14. The method of claim 13, wherein the MLVs also contain polyethylene glycol-conjugated distearoylphosphatidylethanolamine (DSPE-PEG).

15. A method for preparing a hydrophobic drug encapsulated in a cholesterol-free liposome, the method comprising:
suspending one or more of palmitoyloleoylphosphatidylcholine (POPC), dimyristoylphosphatidylcholine (DMPC), and dioleoylphosphatidylcholine (DOPC) in an aqueous buffer to form a lipid suspension,
stirring the lipid suspension for at least 30 minutes at room temperature to form multilamellar vesicles (MLVs),
extruding the MLVs to form large unilamellar vesicles (LUVs) having a diameter of 50 nm to 2 μm,
adding the LUVs to an aqueous solution to form a suspension such that there is substantially no transmembrane potential across the LUVs,
adding a hydrophobic drug to the suspension in the absence of a solubility enhancer to form a mixture,
stirring the mixture for 4 to 48 hours at room temperature to form a drug-loaded liposome suspension, and
filtering the drug-loaded liposome suspension to remove unencapsulated hydrophobic drug,
whereby at least 80% of the added hydrophobic drug is encapsulated in the cholesterol-free liposomes.

16. The method of claim 15, wherein the hydrophobic drug is sirolimus, umirolimus, or everolimus.

17. The method of claim 16, wherein polyethylene glycol-conjugated distearoylphosphatidylethanolamine (DSPE-PEG) is also suspended in the aqueous buffer in the suspending step.

18. A method for treating cancer, the method comprising administering to a subject in need thereof an effective amount of the stable liposomal formulation of claim 1, wherein the effective amount inhibits growth of cancer cells in the subject.

19. A method for treating cancer, the method comprising administering to a subject in need thereof an effective amount of the stable liposomal formulation of claim 3, wherein the effective amount inhibits growth of cancer cells in the subject.

20. A method for treating cancer, the method comprising administering to a subject in need thereof an effective amount of the stable liposomal formulation of claim 10, wherein the effective amount inhibits growth of cancer cells in the subject.
